(19) United States
(12) Patent Application Publication
(10) Pub. No.: US 2007/0129703 A1
(43) Pub. Date: Jun. 7, 2007

(54) Ingestible pressure sensing capsule

(76) Inventors: David T. D'Andrea, Getzville, NY (US); Laura A. Negron, West Seneca, NY (US); John R. Semler, Williamsville, NY (US)

Correspondence Address: PHILLIPS LYTLE LLP;INTELLECTUAL PROPERTY GROUP, 3400 HSBC CENTER, BUFFALO, NY, 14203-3509, US

(21) Appl. No.: 11/294,978

(22) Filed: Dec. 6, 2005

(51) Int. Cl. A61M 31/00

(52) U.S. Cl. 604/503

(57) ABSTRACT

An improved capsule (15) for sensing a contractual force within a mammalian tract, comprising a shell (16), a flexible sleeve (17) affixed to the shell and defining a chamber (19) between the shell and the sleeve, and a pressure sensor (18) operatively arranged to sense pressure within the chamber, whereby a contraction force on the outside of the sleeve produces a corresponding pressure change within the chamber. The chamber may contain a fluid (22) and the fluid may be mineral oil.

### TECHNICAL FIELD

The present invention relates to ingestible capsules and, more particularly, to an ingestible capsule with an improved pressure sensor.

### BACKGROND ART

Ingestible capsules are well known in the prior art and various capsules have been developed. These are generally small pill-like devices that can be ingested or swallowed by a patient. It is known that such capsules may include one or more sensors for determining physiological parameters of the gastrointestinal tract, such as sensors for detecting temperature, pH, pressure and the like. An example of such teachings is found in U.S. Patent Application Publication No. US2003/0191430, the disclosure of which is incorporated herein by reference.

Pressure sensors on capsules in the prior art are generally standard strain gauges or mechanical movement pressure sensors adapted to respond to changes in ambient pressure. However, clinically important physiological contractions of the gastrointestinal tract frequently do not increase ambient pressure in the gastrointestinal tract. Accordingly, there is a need for a pressure sensor on a capsule that more adequately senses gastrointestinal tract contraction forces.

### DISCLOSURE OF THE INVENTION

With parenthetical reference to the corresponding parts, portions or surfaces of the disclosed embodiment, merely for the purposes of illustration and not by way of limitation, the present invention provides an improved capsule (15) for sensing a contractual force within a mammalian tract, comprising a shell (16), a flexible sleeve (17) affixed to the shell and defining a chamber (19) between the shell and the sleeve, and a pressure sensor (18) operatively arranged to sense pressure within the chamber, whereby a contraction force on the outside of the sleeve produces a corresponding pressure change within the chamber. The shell may be rigid and the sleeve may be more elastic than the shell. The chamber may contain a fluid (22) and the fluid may be mineral oil. The chamber may be filled with the fluid to a base pressure and the sensor may sense an increase in pressure in the chamber above the base pressure. The chamber may contain a gas and the gas may be inert. The shell may have an interior surface (23) and an exterior surface (24) and the sensor may be supported by the interior surface of the shell. The sensor may comprise a piezoelectric bridge (26) or an oscillating coil (28) and diaphragm (29). The shell may comprise a fluid port (25) communicating with the chamber and the pressure sensor may sense an increase in pressure in the port. The capsule may comprise a plurality of ribs (20a, 20b) extending from the shell and supporting the sleeve, and the ribs and sleeve may be configured to form a plurality of sub-chambers (21a, 21b), whereby a force on the sleeve produces a corresponding pressure in a sub-chamber that is channeled by the ribs towards the sensor. The sleeve may be stretched over the ribs. The capsule may further comprise a second sleeve (31) affixed to the shell and defining a second chamber (33) between the shell and the second sleeve, and a second pressure sensor (32) operatively arranged to sense pressure within the second chamber, whereby a contraction force on the outside of the second sleeve produces a corresponding pressure change within the second chamber. The sleeve may be attached to the shell by adhesive.

Accordingly, the general object is to provide an improved capsule for determining contraction forces in a mammalian tract.

Another object is to provide an improved capsule that can detect pressure at various points along the outside of the capsule.

Another object is to provide an improved capsule such that physiological tract contraction forces, both amplitude and frequency, are communicated to the pressure sensor element even when there is no change in ambient pressure in the tract.

Another object is to provide an improved capsule with a sensor that optimizes the sensing of contractions in the tract.

Another object is to provide an improved capsule that can be used to measure the flow of force across the surface of the capsule.

Another object is to provide an improved capsule that may be used to orientate the position of the capsule.

These and other objects and advantages will become apparent from the foregoing and ongoing written specification, the drawings and the appended claims.

### BRIEF DESCRIPTION OF THE DRAWINGS

FIG. 1 is a perspective view of the capsule.

FIG. 2 is a longitudinal vertical sectional view of the capsule shown in FIG. 1, taken generally on line 2-2 of FIG. 1.

FIG. 3 is a transverse horizontal sectional view of the capsule shown in FIG. 1, taken generally on line 3-3 of FIG. 1.

FIG. 4 is a perspective view of a second embodiment of the capsule.

FIG. 5 is a longitudinal vertical sectional view of FIG. 4, taken generally on line 5-5 of FIG. 4.

FIG. 6 is a transverse horizontal sectional view of the capsule shown in FIG. 4, taken generally on line 6-6 of FIG. 4.

FIG. 7 is a perspective view of a third embodiment of the capsule.

FIG. 8 is a longitudinal vertical sectional view of the capsule shown in FIG. 7, taken generally on line 8-8 of FIG. 7.

FIG. 9 is a transverse horizontal sectional view of the capsule shown in FIG. 7, taken generally on line 9-9 of FIG. 7.

FIG. 10 is a transverse horizontal sectional view of the capsule shown in FIG. 7, taken generally on line 10-10 of FIG. 7.

### DESCRIPTION OF THE PREFERRED EMBODIMENTS

At the outset, it should be clearly understood that like reference numerals are intended to identify the same structural elements, portions or surfaces, consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

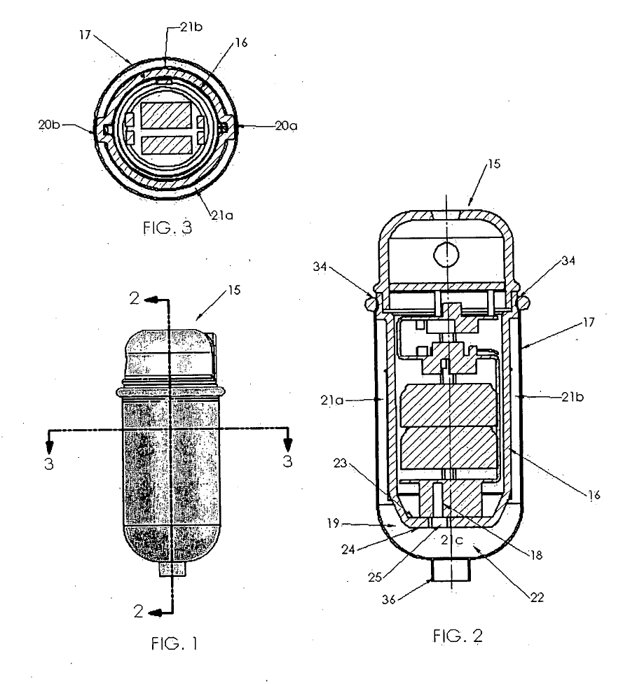

Referring now to the drawings, and more particularly to FIGS. 1-3 thereof, a capsule having an improved pressure sensor is generally indicated at 15. Capsule 15 is shown as being an elongated ellipsoid-shaped device, somewhat resembling a medicament capsule. Capsule 20 is shown as broadly including a shell or casing 16, a flexible sleeve 17 affixed to the shell and defining a chamber 19 between the shell and the sleeve, and a pressure sensor 18 operatively arranged to sense pressure within the chamber and communicating with the chamber through a fluid port 25.

As shown in FIG. 2, the capsule generally has a hard shell or casing 16 which houses the transmitting electronics, battery compartment and sensors. Capsule 15 is adapted to be ingested, implanted, inserted or otherwise positioned within a mammalian body or tract to sense pressure within the body or tract and to transmit such pressure. As shown in FIG. 3, plastic shell 16 is generally a cylindrical member elongated about axis x-x and having generally rounded closed ends. Shell 16 is generally provided to facilitate easy swallowing of the capsule and in the preferred embodiment is composed of a hard polyurethane plastic.

As shown in FIG. 3, shell 16 includes two opposed ribs 20a and 20b extending longitudinally along a portion of the outside cylindrical surface of the shell. Ribs 20a and 20b project radially beyond the outside cylindrical surface of the lower portion of shell 16.

As shown in FIGS. 2 and 3, a sleeve 17 is stretched over ribs 20a and 20b of shell 16 and extends from attachment 34 down around the bottom two-thirds of shell 16. In the preferred embodiment, sleeve 17 does not extend over the entire shell 16 of capsule 15 and is composed of a polyurethane and polycarbonate blend, although sleeve 17 can be made of other elastomeric materials such as natural or synthetic rubber. As shown, sleeve 17 resembles a balloon, and the open end is rolled over to form an annular bead 62 having a slightly smaller inner diameter than the rest of sleeve 17. Bead 62 extends around the outer annular surface of shell 16 at attachment point 34 by interference fit coupling and is secured in place by an adhesive. As shown in FIGS. 1 and 2, the closed bottom end of sleeve 16 includes a small filling port 36 through which chamber 19 between shell 16 and sleeve 17 may be filled with fluid 22.

As shown in FIGS. 2 and 3, sleeve 17 is configured and stretched over sleeve 16 so as to form a chamber 19. Sleeve 17 is adjusted on shell 16 such that the sleeve contacts and is held against ribs 20a and 20b. It is contemplated that this contact may be maintained by stretching the sleeve over the ribs and by the elasticity of sleeve 17, or alternatively the sleeve may be secured to the outer surface of ribs 20a and 20b by glue or other fastening means. Because of the configuration of shell 16, and in particular the use of ribs 20a and 20b, chamber 19 has three connected compartments or sub-chambers 21a, 21b and 21c. Sub-chambers 21a and 21b are defined by the longitudinal space between sleeve 17, shell 16 and ribs 20a and 20b. Sub-chamber 21c is defined by the space between the bottom end of sleeve 17 and the bottom end of shell 16. Sleeve 17 provides a semi-flexible containment area for fluid and translates external force applied to the capsule to pressure sensor 18.

As shown in FIG. 2, shell 16 has an outer surface 24 and an inner surface 23. Sensor 18 is mounted and supported by the interior surface 23 of shell 16. The bottom end of shell 16 includes a fluid port 25 which extends from chamber 21c into the interior of shell 16. Fluid port 25 allows fluid in chamber 21c to communicate with pressure sensor 18.

Pressure sensor 18 is a conventional piezoelectric bridge. As fluid presses against the sensor's bridge, it creates an electric signal which corresponds to the pressure of fluid 22 in chamber 19. Pressure sensor 18 provides good linearity and allows for single point calibration. The GE Nova pressure sensor manufactured by GE Thermal Metrics, of 808 US Highway 1, Edison, N.J., may be used in the preferred embodiment.

Chamber 19 is filled with a fluid 22. In the preferred embodiment, the fluid used is mineral oil. Fluid 22 is a non-compressible medium that forms part of the 360° degree force sensing mechanism for sensor 18. Alternatively, it is contemplated that an inert gas may be used instead of a fluid.

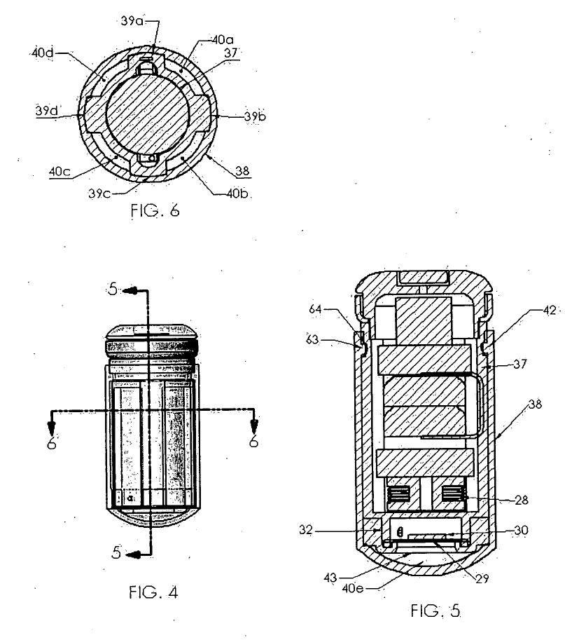

FIGS. 4-6 show a second embodiment of the capsule. In this embodiment, casing 37 has four longitudinally extending ribs 39a-39d, rather than just two ribs. Thus, casing 37 and sleeve 38 form four longitudinally extending sub-chambers 40a-d, which are filled with fluid 22. Sub-chambers 40a-d direct pressure applied at points on the outer cylindrical surface of the capsule towards the bottom end of the capsule and sub-chamber 40e, which is adjacent pressure sensor 32. In this embodiment, sleeve 38 has an open top end and an entirely closed bottom end. The open end of sleeve 38 is attached to casing 37 at attachment 42. Attachment 42 is formed by the mating of an annular ridge 63 at the top peripheral edge of the open end of sleeve 38 and a corresponding annular notch 64 in the surface of casing 37. An adhesive is applied to glue ridge 63 to notch 64. In this embodiment, sleeve 38 is a polyvinyl chloride sleeve and casing 37 is polycarbonate.

Although four ribs are disclosed in this embodiment, it is contemplated that additional ribs may be added. In this embodiment, the ribs are distributed evenly around the circumference of shell 37 and extend about two-thirds of the way up along the outer circumference of shell 37. However, it is contemplated that the ribs may be non-evenly distributed around the circumference of the shell and may extend less than about two-thirds of the way up from the end of the shell. Thus, the length of the ribs may have a length from about 30% to about 100% of the length of shell 38.

In this embodiment, pressure sensor 32 comprises a diaphragm 29 in communication with chamber 41, a non-ferrous disk 30, and an oscillatory coil 28 and capacitor in parallel, which oscillate at a base frequency on the application of a current through coil 28. Diaphragm 29 is supported by the interior surface of shell 37, and an annular rim and a contact port 43 are provided at the end of shell 37. Diaphragm 29 extends across the interior end of port 43. Diaphragm 29 has a flexural modulus that is less then the flexural modulus of sleeve 38 and is capable of deflecting as a result in changing pressure in chamber 40. A non-ferrous disk 30 is attached to the internal surface of diaphragm 29. When diaphragm 29 deflects towards coil 28 as a result of an increase in pressure in chamber 40, non-ferrous disc 29 moves towards coil 28, which decreases the inductance and therefore increases the frequency of oscillation of coil 28. This change is frequency corresponds to a given change in pressure in chamber 40.

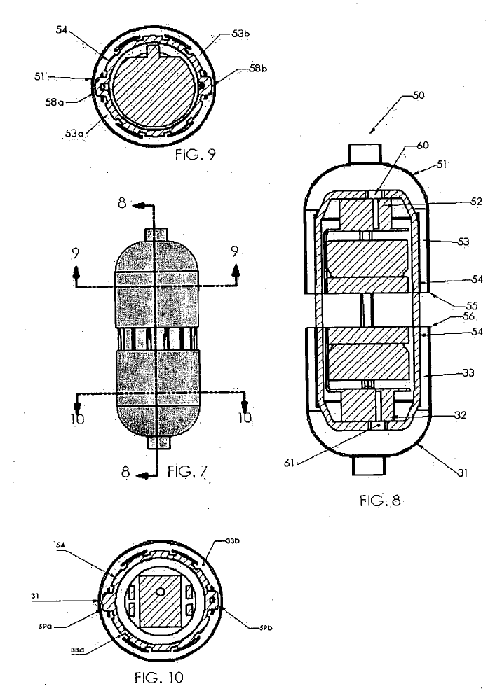

FIGS. 7-10 show a third embodiment 50 of the capsule in which two pressure sensors 32 and 52 are provided at each end of capsule 50. In this embodiment, rather than having one sleeve and multiple ribs extending over two-thirds of the casing, the capsule is provided with opposed sleeves 31 and 51, opposed pressure sensors 32 and 52, and opposed chambers 33 and 53, which are each filled with fluid. In this embodiment, chambers 33 and 53 do not communicate with each other. Although shorter than as provided in embodiment 15, sleeves 31 and 51 are similarly configured, with fill ports at their ends and with annular adhesive attachments 55 and 56, respectively, to shell 54. Shell 54 is configured with ribs that extend from near the middle of capsule 50 towards each end of the capsule to form chambers 33 and 53, respectively. Thus, as shown in FIG. 9, the top portion of the outer circumference of casing 54 includes two longitudinally extending and radially projecting ribs 58a and 58b that separate two sub-chambers 53a and 53b. As shown in FIG. 10, the bottom end of the outer circumference of casing 54 includes two longitudinally extending and radially projecting ribs 59a and 59b that separate two sub-chambers 33a and 33b. Thus, a force on sleeve 51 is translated through fluid in chamber 53 to the top of the capsule and through port 60 to pressure sensor 52. Similarly, a force on sleeve 31 is translated through fluid in chamber 33 to the bottom end of the capsule and through fluid port 61 to pressure sensor 32.

While several forms of the improved capsule with pressure sensor have been shown and described, and various changes and modifications to the apparatus discussed, persons skilled in this art will readily appreciate that various additional changes and modifications may be made without departing from the spirit of the invention, as defined and differentiated by the following claims.

What is claimed is:

1. A capsule for sensing a contractual force within a mammalian tract, comprising:
a shell;
a flexible sleeve affixed to said shell and defining a chamber between said shell and said sleeve; and
a pressure sensor operatively arranged to sense pressure within said chamber;
whereby a contraction force on the outside of said sleeve produces a corresponding pressure change within said chamber.

2. The capsule set forth in claim 1, wherein said shell is rigid and said sleeve is more elastic than said shell.

3. The capsule set forth in claim 1, wherein said chamber contains a fluid.

4. The capsule set forth in claim 3, wherein said fluid is mineral oil.

5. The capsule set forth in claim 3, wherein said chamber is filled with said fluid to a base pressure and said sensor senses an increase in pressure in said chamber above said base pressure.

6. The capsule set forth in claim 1, wherein said chamber contains a gas.

7. The capsule set forth in claim 6, wherein said gas is inert.

8. The capsule set forth in claim 3, wherein said shell has an interior surface and an exterior surface, and said sensor is supported by said interior surface of said shell.

9. The capsule set forth in claim 1, wherein said shell comprises a fluid port communicating with said chamber.

10. The capsule set forth in claim 9, wherein said sensor, said chamber and said port are operatively arranged such that said sensor senses an increase in pressure in said port.

11. The capsule set forth in claim 10, wherein said sensor is a piezoelectric bridge or an oscillator coil and diaphragm.

12. The capsule set forth in claim 1, and further comprising:
a second sleeve affixed to said shell and defining a second chamber between said shell and said second sleeve; and
a second pressure sensor operatively arranged to sense pressure within said second chamber;
whereby a contraction force on the outside of said second sleeve produces a corresponding pressure change within said second chamber.

13. The capsule set forth in claim 1, and further comprising a plurality of ribs extending from said shell and supporting said sleeve.

14. The capsule set forth in claim 13, wherein said ribs are configured to form a plurality of sub-chambers, whereby a force on said sleeve produces a corresponding pressure in a sub-chamber that is channeled by said ribs towards said sensor.

15. The capsule set forth in claim 13, wherein said sleeve is stretched over said ribs.

16. The capsule set forth in claim 1, wherein said sleeve has an open end and a closed end and said open end is attached to said shell with adhesive.
